(19) United States
(12) Patent Application Publication
(10) Pub. No.: US 2024/0342131 A1
(43) Pub. Date: Oct. 17, 2024

(54) Multivitamin for Cystic Fibrosis Patients Undergoing Cystic Fibrosis Transmembrance Conductance Regulator Gene Modulator Therapy

(71) Applicant: Michael Walters, Huntsville, AL (US)

(72) Inventor: Michael Walters, Huntsville, AL (US)

(21) Appl. No.: 18/684,862

(22) PCT Filed: Jul. 14, 2022
(86) PCT No.: PCT/US2022/037053

§ 371 Date: Feb. 20, 2024

Related U.S. Application Data
(60) Provisional application No. 63/233,874, filed on Aug. 17, 2021.
(60) Provisional application No. 63/282,259, filed on Nov. 23, 2021.
(60) Provisional application No. 63/318,864, filed on Mar. 11, 2022.
(60) Provisional application No. 63/318,928, filed on Mar. 11, 2022.

(51) Int. Cl. A61K 31/355; A61K 31/07; A61K 31/122; A61K 31/375; A61K 33/30; A61K 36/534

(52) U.S. Cl. CPC A61K 31/355; A61K 31/07; A61K 31/122; A61K 31/375; A61K 33/30; A61K 36/534

(57) ABSTRACT

The present disclosure provides a multivitamin specifically formulated for patients undergoing CFTR modulator therapy. The multivitamin includes, in various embodiments, pro-vitamin A, vitamin E, a solubilizer, a bile fluid secretion stimulator, vitamin C and a probiotic formulation.

### CROSS REFERENCE TO RELATED APPLICATIONS

This application claims priority to, and the benefit of, pending U.S. Provisional Patent Applications Nos. 63/233,874 filed Aug. 17, 2021; 63/282,259 filed Nov. 23, 2021; 63/318,864 filed Mar. 11, 2022, and 63/318,928 filed Mar. 11, 2022.

### FIELD OF THE DISCLOSURE

The present disclosure pertains to the field of nutritional supplements such as multivitamins. More specifically, the present disclosure pertains to an improved multivitamin for those suffering from Cystic Fibrosis (“CF”) and are undergoing CF transmembrane conductance regulator gene (“CFTR Gene”) modulator formulation (“MF”) therapy.

### BACKGROUND

Cystic Fibrosis is a complex multi-organ disease caused by mutations in the CFTR Gene. The protein encoded by the CFTR Gene, the “CFTR Protein”, forms a CAMP-regulated ion channel responsible for chloride and bicarbonate secretion in epithelial cells. The CFTR Protein also regulates the epithelial sodium channel, playing a role in the humidification of epithelial surfaces. CF typically lowers the life expectancy of those born with it, although new treatments (as discussed herein) are allowing a longer life span for CF patients, but these treatments do have the potential to create some issues with fat-soluble vitamin serum levels with prior art multivitamin formulations based on today's practiced standards of CF care.

One such new treatment is CFTR modulator therapy. MF therapy partially corrects the malfunctioning CFTR protein and depending on the specific MF therapy and the individual patient, offers varying degrees of correction; but low end CFTR function at best. This correction, in addition to improving respiratory function (i.e., FEV1), may improve pancreatic function, reduce fat malabsorption, affect absorption of fat-soluble vitamins from the gastrointestinal (“GI”) tract, and alter liver vitamin stores of certain fat-soluble vitamins. For example, in one case report of a 12-year-old girl, mildly pancreatic insufficient, was prescribed pancreatic enzymes and began taking a multivitamin containing 1,440 IU total vitamin A, of which 432 mcg was preformed vitamin A. Thirty (30) days later, the patient began ivacaftor. Thirteen months later, ivacaftor was replaced with elexacaftor/tezacaftor/ivacaftor (“ETI”). At this time her multivitamin was changed, marginally increasing total vitamin A to 1,553 IU and preformed vitamin A to 436 mcg. Two months after beginning ETI, the ETI and multivitamin were discontinued due elevated intra-cranial pressure. Serum retinol was measured as 48 ug/dl and was within normal limits. Seven weeks later, despite discontinuation of her multivitamin, serum retinol was found to be mildly elevate (57 ug/dl). Given the intake of diet alone, this elevation and progressive increase in serum retinol cannot be explained and supports the suggestion of a release of vitamin A from liver stores. This patient was heterozygous for F-508del. The increased absorption of fat-soluble vitamins, and in particular preformed vitamin A, have the potential to cause vitamin toxicity with prior art formulations.

While there is a plurality of mutations in the CFTR Gene that can cause CF, the current CFTR modulator therapies are mutation specific. The most common CFTR variant in the United States is F508del. Over 85% of individuals in the 2020 Annual CF Foundation Patient Registry who have been genotyped have at least one copy of F508del. These individuals with one copy are called heterozygous for F508del. People with CF who have two copies of F508del are called homozygous and account for about 45% of this population. Patients with two copies tend to have more severe lung disease, characterized by more lung exacerbations, lower FEV1, and lower absorption of fats and fat-soluble vitamins, as well as other disease-related complications. It appears that modulators then have a greater effect on restoring low end CFTR function change—whether a potentiator, corrector, or both a potentiator and corrector—in individuals homozygous for F508del, versus heterozygotes for F508del or another variation in CFTR; and such an effect may be greater when a modulator is started earlier in a patient's life. The effectiveness of modulator therapy may be most pronounced in younger children (e.g., 3 years of age) versus older individuals with CF. (e.g., older teens and persons in their 20's, 30's and 40's), and is subject to the underlying reason for loss of pancreatic function.

To date, the FDA has approved four (4) CFTR modulators, including Kalydeco® (ivacaftor, a potentiator), Orkambi® (lumacaftor/ivacaftor, a corrector and potentiator combination more potent than Kalydeco), SymdekoR (tezacaftor/ivacaftor, a corrector and potentiator even more potent than both Kalydeco and Orkambi) and Trikafta® (elexacaftor/tezacaftor/ivacaftor, two correctors and one potentiator. Trikafta is the most effective MF therapy in restoring CFTR function approved to date by FDA.

Ivacaftor, the first MF therapy approved by FDA works by opening the gates of the CFTR protein and helps to keep those gates open longer. Tezacaftor and Elexacaftor help the CFTR protein form the correct shape so the CFTR protein can get to the surface of the cell, where the protein is normally located. Ivacaftor (Kalydeco), Lumacaftor/Ivacaftor (Orkambi) and Tezacaftor/Ivacaftor (Symedco) MF therapies are only moderately effective, resulting in an average of a 2% increase in lung function (as measured by FEV1). Elaxacaftor/Tezacaftor/Ivacaftor (Trikafta) (“ETI”), a triple combination of modulators, and the most recent market introduction by VERTEX, brings many more CFTR gates to the right place in the cell, and increases FEV1 by an average of 12% in patients with CF. This triple MF therapy, with two correctors and one potentiator, allows many more chloride ions to move in and out of the cell and restores low-end function of the CFTR protein to a greater degree that previous MF therapies. It is this increased effectiveness that has brought attention to the subject of prior art multivitamins in the supportive care of CF, and concerns surrounding elevated fat-soluble vitamin serum levels and the potential for toxicity with the advances of MF therapy.

While the majority of interest in CFTR modulator therapy has focused on the airway passages (e.g., the lungs), due to chronic airway obstruction, inflammation and infection—as this results in progressive damage to the lungs, and accounts for about 80% of morbidity and mortality in CF—the CFTR Protein is also absent or functionally impaired on intestinal, biliary and pancreatic epithelium in CF, affecting the body's ability to absorb essential nutrients from the gastrointestinal tract (“GI tract”). The PROMISE Study, a prospective, multi-center observational study of some 180 patients, sought to measure the clinical effectiveness of ETI in children 6-11 years of age, with one or more copies of the F508 deletion. The study included patient-reported outcomes and basic quality-of-life measures diabetes, bone health, microbiology, lung function, gastrointestinal health, blood sugar and liver function. The protocol included one measurement before ETI therapy and 4 subsequent measurements-one month, 6 months, 12 months, and 24 months. A reported adverse event with ETI usage is weight gain. Weight gain is directly related to the improvement in absorption of dietary fats. This increased absorption of fats likely also results in an increased absorption of fat-soluble vitamins, an increase that is variable based on the MF therapy prescribed, the underlying cause resulting in loss of pancreatic function, and the individual patient. A measure not included in the PROMISE Study was serum fat-soluble vitamin levels.

No large placebo-controlled study or published data is available to evaluate of the effects of MF Therapy on fat-soluble serum levels, nor has the CF Foundation expressed any concern for patients taking prior art CF multivitamin formulations on MF therapies; and to the contrary, has expressed that it is “too early to draw any conclusions” around MF Therapy and serum vitamin levels with prior art CF multivitamins.

About 85% of patients with CF are pancreatic insufficient or suffer from pancreatic insufficiency (“PI”) due to CFTR dysfunction, resulting in obstruction of the pancreatic duct by thick mucus and destruction of the exocrine pancreatic tissue that produces lipases, proteases and amylases involved in the digestion of food. Once a diagnosis of PI is made, pancreatic enzyme therapy (“PERT”) is often initiated. Despite PERT intervention, malabsorption of fats and fat-soluble vitamins (A, D, E, K) remains an ongoing manifestation. Historical guidance has indicated that certain multivitamin formulations could be beneficial for the treatment of CF patients, and they are widely used. However, these prior art vitamins have not been formulated with our consideration of the recent advancements of MF therapy in mind and, accordingly, there is a need in the prior art for a change to such multivitamin formulations. That multivitamin formulation change is provided by the present disclosure.

### DESCRIPTION OF DRAWINGS

To further illustrate the advantages and features of the present disclosure, a more particular description of the invention will be rendered by referenced to specific embodiments thereof which are illustrated in the appended drawings. It is appreciated that these drawings are not to be considered limiting in scope. The invention will be described with additional detail through the use of the accompanying drawings in which:

FIGS. 1A and 1B show one embodiment of the prior art multivitamins staining on different fabric swatches after individual treatments with peroxide and bleach.

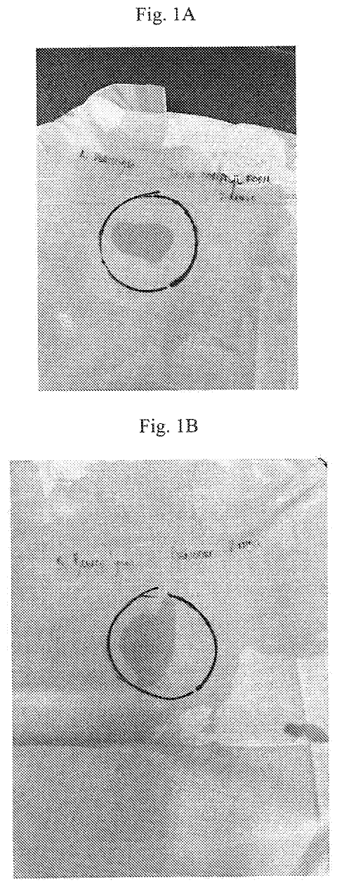

FIGS. 2A and 2B show one embodiment of the multivitamin of the present disclosure not leaving stains on different fabric swatches after individual treatments with peroxide and bleach.

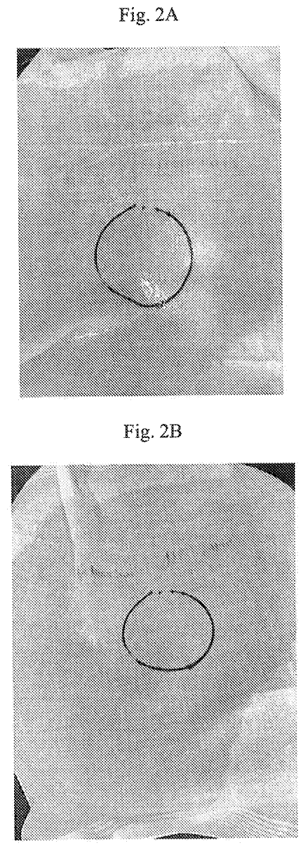

### DETAILED DESCRIPTION

The present disclosure provides a multivitamin specifically formulated for patients undergoing CFTR modulator therapy. The multivitamin includes, in various embodiments, pro-vitamin A (or beta-carotene), vitamin E, a solubilizer, a bile fluid secretion stimulator, high dose vitamin C and a probiotic formulation. Also to be considered within the scope of this embodiment is a multivitamin containing at least two of pro-vitamin A, vitamin E, a solubilizer, a bile fluid secretion stimulator, high dose vitamin C and a probiotic formulation. Also to be considered within the scope of this embodiment is a multivitamin containing at least three of pro-vitamin A, vitamin E, a solubilizer, a bile fluid secretion stimulator, high dose vitamin C and a probiotic formulation.

Also, to be considered within the scope of this embodiment is a multivitamin containing at least two of pro-vitamin A, vitamin E, a solubilizer, a bile fluid secretion stimulator, high dose vitamin C and a probiotic formulation.

CF causes PI and reduces intestinal absorption of fats and fat-soluble vitamins such as vitamins A, D, E, and K. PI is present in up to 85% of CF patients at birth. CFTR Modulator Therapy leads to a rescue of CFTR function of about 2% to 12%. This is comparable to the lower range of CFTR function in patients with CF and residual function mutations.

One aspect of this invention in one embodiment is to decrease the preformed vitamin A content in the formulation of the vitamin from what is taught in the prior art, and to increase pro-vitamin A content. A vitamin is an organic molecule that is an essential micronutrient which an organism needs in small quantities for the proper functioning of overall metabolism. Vitamin A, a fat-soluble vitamin, acts as a regulator of cell and tissue growth and cell differentiation and is an essential nutrient. Vitamin A is a group of compounds that includes retinol, retinal, and retinoic acid, all preformed vitamin A; and several pro-vitamin A carotenoids, most notably beta-carotene.

Preformed vitamin A is known to be insatiably absorbed and increases the propensity for toxicity (e.g., hypervitaminosis A). In this embodiment, the amount of provitamin A (beta-carotene) per serving for persons over three (3) years of age is increased to a total vitamin content of 6000 mcg, with preformed vitamin A reduced to 3.5% to 5.5% and approximately 210 mcg to 330 mcg of total vitamin A content. Current prior art formulations range from 8% and 436 mcg to 12% and 576 mcg of preformed vitamin A. For newborns and infants up to 3 years of age, total A content is increased to 2000 mcg per 1 ml of volume, with preformed vitamin A content reduced to 7% to 8% of the total vitamin A content and to approximately 140 mcg to 160 mcg. Current formulations range from 13% and 225 mcg to 25% and 347 mcg of preformed vitamin A. The increase in pro-vitamin A would also likely fully accommodate individuals with genetic variation in the BCMO1/BCO1 gene, as substitution with a T allele on both rs12934922 and rs7501331 results in 69% decreased conversion of beta-carotene to retinol. In an alternate embodiment, total Vitamin A is not increased, but preformed vitamin A remains between approximately 210 mcg and 336 mcg per serving for those over three (3) years of age, and approximately 140 mcg to 160 mcg per 1-2 ml for newborns and infants up to three (3) years of age.

The various forms of vitamin A are solubilized into micelles in the intestinal lumen and absorbed by duodenal mucosa. Preformed vitamin A (retinyl acetate, retinyl palmitate) is absorbed from the digestive tract via passive diffusion, and insatiably absorbed. Pro-vitamin A is taken up by enterocytes by the membrane transporter protein scavenger receptor B1 (SCARB1), which is upregulated in times of vitamin A deficiency. If vitamin A status is within the normal range, SCARB1 is downregulated, reducing conversion and absorption of pro-vitamins A. Also downregulated is the enzyme beta-carotene 15, 15′ monooxygenase, coded for by the BCMO1 gene, responsible for cleaving beta-carotene in retinal.

Overconsumption of preformed vitamin A can be highly toxic and is especially contraindicated prior to and during pregnancy, as it can result in severe birth defects. The UL for preformed vitamin A in adults is 3000 mcg. There is no UL for beta-carotene.

While multivitamin supplement formulations that contain beta-carotene to lower preformed vitamin A content offer clear safety advantages to individuals, beta-carotene does present a challenge-staining. In a liquid form, beta-carotene stains clothing when it comes in contact with the material. The stain is difficult, if not impossible to remove. When used in a chewable multivitamin, beta-carotene can stain teeth.

Early multivitamins used in CF care contained only preformed vitamin A. A study was conducted by Brei (Clin Nutr. 2013 October; 32 (5): 805-10) to assess vitamin status and appropriate supplementation in patients receiving vitamin A. The Brei study found (a) that the range of the supplementation dosage was 0-20,000 IU/day, (b) 25% of patients did not need any vitamin A supplementation, and (c) total vitamin A intake exceeded the recommended Upper Limit (“UL) in 69% of studied subjects. This, and other findings by Graham-Maar (Am J Clin Nutr. 2006 July; 84 (1): 174-82) led to multivitamin formulations that included both pro-vitamin A and preformed vitamin A to minimize this potential for toxicity.

Vitamin A is obtained from the diet as either preformed vitamin A or as pro-vitamin A carotenoids. Total vitamin A intake in the US averages approximately 600 mcg per day, of this about 70-75% is thought to be due to the intake of preformed vitamin A in eggs, meat, butter, milk, and fortified foods. The recommended daily allowance of vitamin A is (i) 900 mcg for men 14 and older and (ii) 700 mcg for women 14 and older, (with Upper Limits (“UL”) of 2000 mcg for ages 14-18 and 3000 mcg for those over 18 years of age), (iii) 9-13 years of age, 600 mcg, (with a UL of 1700 mcg), (iv) 400 mcg for children 4-8 years, (with a UL of 900 mcg), (v) 300 mcg for 1-3 years of age, (with a UL of 600 mcg), (vi) 500 mcg for ages 7-12 months, (with a UL of 600 mcg), and (vii) 400 mcg for 0-6 months, (with a UL of 600 mcg). CF prior art multivitamins often contain significantly higher amounts of preformed vitamin A than the age-specific recommended daily allowance (“RDA”), independent of amounts of preformed vitamin A from dietary sources. While MF therapy helps to restore low end CFTR function, the variability of restoration of CFTR function based on the choice of MF, varied individual patient response due to underlying cause of loss of pancreatic function, differences in dietary intake of preformed vitamin A in patients, and the knowledge that beta carotene is only converted to an active form of vitamin A as needed, one aspect of this invention introduces a significant change from prior art formulations in both total vitamin A content and the percentage of preformed and pro-vitamin A forms to overcome risks of toxicity in patients receiving MF therapy.

Additionally, vitamin E plasma levels are likely affected by CFTR modulator therapy. One potential cause of this reduction may be the induction of cytochrome P450 system; Another is an increase in bile production. A review of early studies with MF therapy shows inconsistent results in the changes seen in vitamin E levels in patients with CF:
Study #1: No significant change in serum vitamin E levels. Heterozygous for F508del. MF therapy was ivacaftor. Duration: 3 months.
Study #2: Significant decrease in serum vitamin E levels at year one and year two with lumacaftor/ivacaftor. All patients homozygous F508del.
Study #3: Increased vitamin E levels, a change positively correlated with treatment months-treatment ranged from 9 to 29 months. 27 patients were homozygous F508del; 28 patients were heterozygous F508del. Before MF therapy of lumacaftor/ivacaftor, homozygous F508del had significantly lower vitamin E serum levels than heterozygous F508del.

While no publicly available data is available on the effects of tezacaftor/lumacaftor/ivacaftor on the effects on vitamin E serum levels, it is likely that:
1. Changes in CFTR function with MF therapies are not isolated to the lung. These changes are also realized—to some and varying degrees—in intestinal, biliary, and pancreatic epithelium.
2. The restorative function of the CFTR protein when ETI is prescribed, versus earlier and other MF therapies, is greater and clinically relevant in both individuals heterozygous and homozygous for F508del, a finding that appears to differ than when lumacaftor/ivacaftor or ivacaftor are prescribed.

Accordingly, another aspect of this invention, in one embodiment, is to decrease the amount of vitamin E in the multivitamin to approximately 75-100 IU (50.25-67 mcg) per serving for those over 3 years of age; recognizing that “the degree of effect” of MF therapy on vitamin E serums levels is a function of potency of the MF therapy, the homozygous versus heterozygous F508del status of the individual, and duration of MF therapy. For newborns and infants up to 3 years of age, vitamin E is reduced by approximately 50%, from 50-100 IU (33.5-67 mcg) in prior art multivitamins, to 25-50 IU (16.75-33.5 mcg).

Current prior art CF-specific multivitamins for those over 3 years of age include 150 IU (100 mcg) to 200 IU (134 mcg) per serving in their formulations. Of note, additional vitamin E is included as mixed tocopherols or a tocopherol rich extract, referenced in the Other Ingredients section of the label and not included in the total vitamin E content in the label in prior art multivitamins. This serves as an additional source and amount of Vitamin E, despite the Supplement Facts label disclosure of just 150-200 IUs per serving for those over 3 years of age. In one embodiment, the amount of vitamin E may be between about 75-100 IUs per serving of the multivitamin of total vitamin E intake for those over 3 years of age. For newborns and infants, an amount of 25-50 IUs and an approximately 50% reduction in vitamin E content when compared to prior art formulations. While it is anticipated that individuals who are homozygous F508del will realize greater increases in serum vitamin E levels when prescribed MF therapy, it is also believed that with a shift in prescribing to ETI—the most potent modulator approved by FDA to date and commercially available—this reduced vitamin E content will provide the needed therapeutic serum levels for both individuals homozygous and heterozygous for F508del on MF therapy.

The vitamin of the present disclosure, in one embodiment, also includes a solubilizer. While the prior art CF multivitamins often contain emulsifiers, they do not contain solubilizers. An understanding of the difference between an emulsifier and a solubilizer is important to understanding this invention.

Emulsifiers enable the suspension of oil in water; they are not water soluble. This results in large oil droplets suspended in an aqueous medium, like fats and fat-soluble vitamins within the contents in the small intestine. Emulsification of fats is a critical part of digestion and promotes micelle formation in the presence of bile salts. Most of the fluid in the digestive tract is water-based, so when one consumes fat, digested fat tends to form large globules in the intestine. Large globules are incompletely and inefficiently absorbed and limit the absorption of fat-soluble vitamins. In individuals without CF, emulsification is promoted by bile; in CF, however, bile composition differs from individuals without CF, the bile pool is reduced due to CFTR function in the gall bladder, and complete or partial obstruction of the bile duct that can occur. This inefficiency may explain why a significantly higher intake of fat-soluble vitamins (A, D, E, K) has been required in patients with CF, than individuals without CF; and despite these higher intakes, why many CF patients remain low fat-soluble vitamin status, despite ongoing increases in fat-soluble vitamin intake over the last 20+ years; and why PERT fails to fully normalize fat digestion and absorption.

Solubilizers, on the other hand, help to make otherwise insoluble materials soluble in an aqueous medium, like fats and fat-soluble vitamins in water. Solubilization enhances the solubility of fatty acids by several magnitudes (100 to 1000-fold) and absorption is achieved by the formation of mixed micelles. The diameter of emulsified lipid droplets ranges from 100 to 1000 nm, whereas the diameter of mixed micelles ranges from 3 to 5 nm. Solubilization is necessary for hydrophobic components to efficiently overcome the diffusion barrier of the unstirred water layer of the cells lining the small intestines (i.e., enterocytes), which separate the enterocytes from the luminal contents of the intestine, and to more efficiently, and more completely absorb fats and support the absorption of fat-soluble vitamins-notably the products from long chain fatty acids.

Accordingly, the multivitamin includes a solubilizer. In one embodiment, the solubilizer is a water soluble solubilizer. In one preferred embodiment, the water soluble solubilizer are cyclodextrins (e.g., alpha-cyclodextrin, beta-cyclodextrin, beta-cyclodextrin derivatives) or polyoxymethylene (20) sorbitan monolaurate (“Polysorbate 20”). In an alternative embodiment, the solubilizer is a water miscible solubilizer such as glycols, which include glycerin USP, propylene glycol, and polyethylene glycol “PEG”.

The multivitamin of the present disclosure also includes a bile fluid secretion stimulator, such as peppermint oil (“PO”) in one embodiment. Bile acids, the essential components of bile, are involved in some critical physiological functions such as intestinal digestion and supporting lipid absorption. Therefore, they contribute to digestive function and the prevention of disease by promoting bile secretion and raising bile acid content. PO a natural source of vegetable oil that mainly contains L-menthol, L-menthone and isomenthone, stimulates bile fluid secretion and thus has a choleretic effect. This effect may be the result of upregulating CYP7A1 and FXR mRNA levels, suggesting that the molecular mechanisms are related to gene expression involved in bile acid synthesis.

In addition, while PO may be used as a flavoring (i.e., to taste mask) in foods and beverages, no prior art CF-specific multivitamins include PO to stimulate bile production. The presence of bile salts is required to support the formation of micelles and facilitate the absorption of fats and fat-soluble vitamins.

The multivitamin of the present disclosure also includes high dose vitamin C in one embodiment in the range of approximately 350 mg to 1200 mg daily which serves, at least in part, to induce the opening of the CFTR Chloride channels and enhance epithelial secretions to reduce the stickiness of the mucus on intestinal epithelia in the gut. Prior art multivitamins for those over 3 years of age include 70-100 mg of vitamin C per serving for individuals over 3 years of age, and 45 mg for newborns to 12 months and 90 mg for 1-3 years of age. This novel formulation developed to be used for those on MF therapies, significantly increases vitamin C content with the design to increase the opening of CFTR channels and aid in the support of absorption. In a preferred embodiment, vitamin C is in a liposomal form. Alternatively, in another preferred embodiment, vitamin C may be from ascorbic acid or a combination of ascorbic acid and zinc ascorbate. The recommended daily allowance (“RDA”) for vitamin C, in those 19 years of age and older is 90 mg daily for men and 75 mg for women. The UL for adults is 2000 mg. For (i) 14-18 years of age, the RDA is 75 mg and 65 mg for males and females respectively (1800 mg UL); (ii) 45 mg for individuals 9-13 (1200 mg UL); (iii) children 4-8 years of age 25 mg (650 mg UL); and (iv) children 1-3, 15 mg (400 mg UL). There is no established UL for newborns and infants from 0 to 12 months.

Vitamin C is a biological regulator of CFTR-mediated Cl secretion in the epithelia. While focus has previously been directed to the nasal epithelium, vitamin C can theoretically activate Cl transport in the GI tract at appropriate concentrations. At the appropriate concentrations, vitamin C induces the openings of the CFTR Chloride channels-increasing its average open probability from zero to 0.21 (+0.08)—enhancing epithelial secretions and reducing the stickiness and viscosity of mucus on the surface of the intestinal epithelia. This concept is further supported by the fact that high doses of vitamin C can cause secretory diarrhea as a side effect, which is readily explained by its stimulatory effect on CFTR-mediated Cl secretion in intestinal epithelial cells. Vitamin C crosses the small intestine, and then enters the plasma. We suggest that levels of vitamin C of from 150 mg to 200 mg per 1 ml serving in newborns and infants, and from 400 mg to 600 mg per serving in those 3 years of age and older per serving, that when taken orally as a single dose, can exert influence over CFTR Cl channels and support the uptake of fat-soluble vitamins (A, D, E, K) by inducing the opening of CFTR CI channels of epithelial cells bordering the CF small intestine, without a detectable increase in cAMP.

The multivitamin disclosed herein optionally includes zinc as well. In one embodiment for those over 3 years of age the multivitamin contains about 10 mg zinc per serving with the zinc being from either zinc oxide or zinc ascorbate. In another embodiment for those under 3 years of age, the multivitamin contains about 5 mg zinc as zinc oxide.

CF patients often suffer from abnormal gut flora. Accordingly, in one embodiment, the multivitamin of the present disclosure provides a probiotic formulation to help correct this abnormality. In one embodiment, the probiotic formulation includes at least one of Lactobacillus rhamnosus GG, Lactobacillus retuteri, Lactobacillus rhamnosus and Bifidobacterium animalis. In an alternate embodiment, the multivitamin of the present disclosure includes at least two (2) of the forgoing.

The multivitamin of the present disclosure can be formulated for human use and may comprise and may, optionally include a pharmaceutically acceptable carrier. Additionally, methods of treatment of patients in need of treatment or in need of prevention of the side-effects of modulator therapy, such as those discussed herein, are within the scope of this disclosure.

The term “individual” or “patient” as used herein refers to any animal, including mammals, such as, but not limited to, mice, rats, other rodents, rabbits, dogs, cats, swine, cattle, sheep, horses, primates, or humans.

The term “in need of prevention” as used herein refers to a judgment made by a caregiver that a patient requires or will benefit from prevention. This judgment is made based on a variety of factors that are in the realm of a caregiver's expertise and may include the knowledge that the patient may become ill as the result of a disease state that is treatable by a compound or pharmaceutical composition of the disclosure.

The term “in need of treatment” as used herein refers to a judgment made by a caregiver that a patient requires or will benefit from treatment. This judgment is made based on a variety of factors that are in the realm of a caregiver's expertise and may include the knowledge that the patient is ill as the result of a disease state that is treatable by a compound or pharmaceutical composition of the disclosure.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

The terms “about” and “approximately” shall generally mean an acceptable degree of error or variation for the quantity measured given the nature or precision of the measurements. Typical, exemplary degrees of error or variation are within 20 percent (%), preferably within 10%, more preferably within 5%, and still more preferably within 1% of a given value or range of values. Numerical quantities given in this description are approximate unless stated otherwise, meaning that the term “about” or “approximately” can be inferred when not expressly stated. As used herein, the singular forms “a”, “an” and “the” are intended to include the plural forms as well, unless the context clearly indicates otherwise.

1. A multivitamin for a patient over three (3) years of age, the multivitamin comprising:
a. vitamin A
b. vitamin E; and
c. a solubilizer.

2. The multivitamin of claim 1 wherein the solubilizer is a water soluble solubilizer.

3. The multivitamin of claim 1 wherein the solubilizer is a water miscible solubilizer.

4. The multivitamin of claim 1 further comprising a bile fluid secretion stimulator.

5. The multivitamin of claim 4 wherein the bile fluid secretion stimulator is peppermint oil.

6. The multivitamin of claim 1 further comprising at least two (2) of vitamin C, vitamin K and zinc.

7. The multivitamin of claim 5 further comprising at least two (2) of vitamin C, vitamin K and zinc.

8. A liquid multivitamin for a patient under three (3) years of age, the multivitamin comprising:
a. vitamin A;
b. vitamin E; and
c. a solubilizer.

9. The multivitamin of claim 8 wherein the solubilizer is a water soluble solubilizer.

10. The multivitamin of claim 8 wherein the solubilizer is a water miscible solubilizer.

11. The multivitamin of claim 8 further comprising a bile fluid secretion stimulator.

12. The multivitamin of claim 11 wherein the bile fluid secretion stimulator is peppermint oil.

13. The multivitamin of claim 8 further comprising at least two (2) of vitamin C, vitamin K and zinc.

14. The multivitamin of claim 12 further comprising at least two (2) of vitamin C, vitamin K and zinc.

15. A multivitamin comprising:
a. preformed vitamin A;
b. pro-vitamin A; and
c. a solubilizer.

16. The multivitamin of claim 8 wherein the solubilizer is a water soluble solubilizer.

17. The multivitamin of claim 8 wherein the solubilizer is a water miscible solubilizer.

18. The multivitamin of claim 8 further comprising a bile fluid secretion stimulator.

19. The multivitamin of claim 11 wherein the bile fluid secretion stimulator is peppermint oil.

20. The multivitamin of claim 8 further comprising at least two (2) of vitamin C, vitamin K and zinc.
